(19) United States
(12) Patent Application Publication
(10) Pub. No.: US 2024/0285446 A1
(43) Pub. Date: Aug. 29, 2024

(54) Sanitary Knickers

(71) Applicant: Huixiong Paoxiao (Shanghai) Network Technology Co., Ltd., Shanghai (CN)

(72) Inventor: Lida Zhou, Shanghai (CN)

(21) Appl. No.: 18/175,453

(22) Filed: Feb. 27, 2023

(51) Int. Cl. A61F 13/496

(52) U.S. Cl. CPC A61F 13/496

(57) ABSTRACT

A sanitary knicker includes an underpants body having a first coverage area and a second coverage area at a rear end thereof, the first coverage area is used for fitting a user's left hip, the second coverage area is used for fitting the user's right hip; the sanitary knicker also comprising a middle crotch cloth, a front end thereof is disposed at a front end of the underpants body, a rear end thereof extends toward the rear end of the underpants body to connect with the first coverage area and the second coverage area to wrap a part of the user's left hip, a part of the user's right hip and an area there-between.

### FIELD OF THE INVENTION

The present invention relates to women's clothing used in a menstrual period, particularly, to a reusable sanitary product for women during the menstrual period, especially, to a sanitary knicker.

### BACKGROUND OF THE INVENTION

A sanitary knicker is a sanitary article used by women to absorb menstrual blood discharged from their bodies during their menstrual periods and prevent their clothes from being wet and dirty by menstrual blood. In the early period, toilet paper is used by women during their menstrual periods. Now, with economic development, sanitary napkins, sanitary towels, or menstrual pants have been developed to absorb menstrual blood. With the change in women's social roles and the needs of various occasions, more and more women choose sanitary knickers.

Chinese Patent No. CN212697697U discloses a kind of sanitary knicker, which comprises a trouser body sewn by a front piece, a rear piece and a crotch piece. The outer wall of the crotch piece is fixedly connected with two groups of fixing pieces symmetrically arranged on both sides thereof. A through hole is formed between the fixing pieces and the crotch piece. The rear piece is fixedly connected with an elastic belt, and one free end of the elastic belt runs through the through hole and is clamped with the front piece.

Although the sanitary knicker in the above patent can limit the sanitary towel so that the sanitary towel can always fit the human body, thus reducing the occurrence of side leakage, the suture lines between the front piece, the rear piece and the crotch piece are easily product friction with human skin, leaving traces on the skin, thus reducing the comfort of wearing. In addition, the crotch piece is used to fit the sanitary towel, which can not directly absorb menstrual blood discharged from bodies and which does not extend to the rear waist so that the crotch piece in the middle cannot be completely covered so that the sanitary knicker is easily side leakage when women use them in menstrual period.

### SUMMARY OF THE INVENTION

The objective of the present invention is to provide a sanitary knicker to solve the technical problems that the crotch piece in the sanitary knicker in the prior art can not directly absorb menstrual blood discharged from bodies, and is not completely covered the middle part in the crotch portion in the sanitary knicker because of the structure limitation thereof so that the sanitary knicker is easily side leakage when women use them in menstrual period.

For achieving the above objective, the embodiment of the present invention provides the sanitary knicker, which includes an underpants body having a first coverage area and a second coverage area at a rear end thereof, the first coverage area is used for fitting a user's left hip, the second coverage area is used for fitting the user's right hip. The sanitary knicker also has a middle crotch cloth, a front end of the middle crotch cloth is disposed at a front end of the underpants body, a rear end thereof extends toward the rear end of the underpants body to connect with the first coverage area and the second coverage area to wrap a part of the user's left hip, a part of the user's right hip and the area there-between.

Optionally, an inner surface of the middle crotch cloth has an absorption portion, comprising an absorption layer, a locking water layer and a leak-proof layer, overlapped together. The absorption layer is used to inhale the user's menstrual blood, the locking water layer is used to adsorb the user's menstrual blood, and the leak-proof layer is used to isolate the menstrual blood in the locking water layer with other clothing.

Optionally, the absorption layer, the locking water layer and the leak-proof layer are arranged from a side closing to the user's skin to the outside; the absorption layer, the locking water layer and the leak-proof layer are in an integrated structure.

Optionally, the underpants body includes a front-left crotch cloth, a front-right crotch cloth and a rear-left crotch cloth and a rear-right crotch cloth; the front-left crotch cloth is connected with the rear-left crotch cloth, and the front-right crotch cloth is connected with the rear-right crotch cloth; the front-left crotch cloth and the front-right crotch cloth are used to fit the user's abdomen, the rear-left crotch cloth is used to fit a part of the user's left hip, and the rear-right crotch cloth is used to fit a part of the user's right hip.

Two sides of the front end of the middle crotch cloth are respectively connected with the front-left crotch cloth and the front-rear crotch cloth, and two sides of the rear end of the middle crotch cloth are connected with the rear-left crotch cloth and the rear-right crotch cloth respectively.

Optionally, the front-left crotch cloth, the front-right crotch cloth, the rear-left crotch cloth, and the rear-right crotch cloth are all adhesive with the middle crotch cloth together.

Optionally, a left side of the rear-left crotch cloth extends to the front-left crotch cloth and is connected with it, and a right side of the rear-left crotch cloth is connected with a left side of the rear end of the middle crotch cloth.

Optionally, a right side of the rear-right crotch cloth extends to the front-right crotch cloth and is connected with it, a left side of the rear-right crotch cloth is connected with a right side of the rear end of the middle crotch cloth.

Optionally, the rear-left crotch cloth and the rear-right are symmetrically disposed on the underpants body; the front-left crotch cloth, the front-right crotch cloth, the rear-left crotch cloth, and the rear-right crotch cloth are all in sheet structure.

Optionally, the sanitary knicker also includes a waistcloth, and the waistcloth is connected to all of the front-left crotch cloth, the front-right crotch cloth, the rear-left crotch cloth, the rear-right crotch cloth and the middle crotch cloth, and they are connected by an adhesive way. The waistcloth is provided with many air holes.

One or more of the technical schemes in the embodiments in the present invention have at least one of the following technical effects:

In the sanitary knicker according to the embodiments, the rear end of the middle crotch cloth extends toward the rear end of the underpants body to connect with the first coverage area and the second coverage area to wrap a part of the left hip, a part of the right hip and the area there-between, and the rear end of the middle crotch cloth covers the area between the left hip and the right hip and reaches the waist, resolving the deficiencies that the traditional middle crotch cloth cannot cover the waist, which increases the absorption area of the middle crotch cloth, improves the absorption effect and leakage prevention effect of the middle crotch cloth, makes the user's skin in a fresh and cool state.

In the sanitary knicker according to the embodiments, the underpants body and the middle crotch cloth are adhered in an integrated structure, replacing the traditional way of sewing the underpants body and the middle crotch cloth through needle and thread, so that there is no suture line between the underpants body and the middle crotch cloth, reducing the friction between the suture line on the user's skin, and making the sanitary knicker more comfortable to wear.

In the sanitary knicker according to the embodiments, the absorption layer, the locking water layer and the leak-proof layer are bonding to form the absorption portion, which can absorb the user's menstrual blood and prevent the menstrual blood from being leakage, thereby achieving the effect of leakage prevention. The users do not need to use disposable sanitary products such as sanitary napkins and cotton slivers during their menstrual period anymore, they can wear the sanitary knicker directly, thus improving the practicability of the sanitary knicker.

In the sanitary knicker according to the embodiments, the waistcloth is provided with many air holes, the air holes can breathe and sweat at the waist, improving the comfort of wearing, and avoiding the phenomenon of sultry on the waist.

### BRIEF DESCRIPTION OF THE DRAWINGS

In order to more clearly explain the technical schemes in the embodiments of the present invention, the following will briefly introduce the embodiments or the drawings needed in the description of the prior art. It is obvious that the drawings in the following description are only some embodiments of the present invention, For those skilled in the art, other drawings may also be obtained from them without creative labor.

FIG. 1 is a perspective view of the sanitary knicker according to the embodiments in the present invention.

FIG. 2 is a rear view of the sanitary knicker according to the embodiments in the present invention.

FIG. 3 is an inner structure view of the sanitary knicker according to the embodiments in the present invention.

FIG. 4 is a perspective view of the absorbing part of the sanitary knicker according to the embodiments in the present invention.

FIG. 5 is a front view of the sanitary knicker according to the embodiments in the present invention.

FIG. 6 is a perspective view of the waistcloth of the sanitary knicker according to the embodiments in the present invention.

### DETAILED DESCRIPTION OF ILLUSTRATED EMBODIMENTS

The embodiments of the present invention will be described in detail below. Examples of the embodiments are shown in the drawings, where the same or similar reference numerals throughout indicate the same or similar elements or elements having the same or similar functions. The embodiments described below by referring to the accompanying drawings are exemplary and are intended to be used to explain the embodiments of the present invention, but cannot be understood as limiting the present invention.

In the description of the embodiment of the present invention, it needs to be understood that the azimuth or position relation indicated in the terms “length”, “width”, “upper”, “lower”, “front”, “rear”, “left”, “right”, “vertical”, “horizontal”, “top”, “bottom” inside”, “outside” and so on are based on the azimuth or position relation shown in the attached drawing, only for the convenience of describing the embodiment of the present invention and simplifying the description, rather than indicating or implying that the device or element must have a specific orientation, with a specific orientation and operation, so it can not be understood as a limitation to the present invention.

Furthermore, the terms “first”, “second” are used only for descriptive purposes and it can not be understood as indicating or implying relative importance or implicitly the number of technical features indicated. Thus, the qualification of “first” and “second” features may include one or more of them explicitly or implicitly. In the description of the embodiments of the invention, the meaning of “multiple” is two or more, unless otherwise specified.

In the embodiment of the invention, unless other specified and qualified, the terms “installation”, “connection”, “fixed” and other terms shall be broadly understood, for example, it may be fixed connection, detachable connection, or integration; it may be a mechanical connection or an electrical connection; it may be directly connected or indirectly connected by intermediate media, it may be an internal connection of two components or the interaction of two components. For ordinary technicians in the field, the specific meaning of the above term in the embodiment of the invention can be understood according to the specific circumstances.

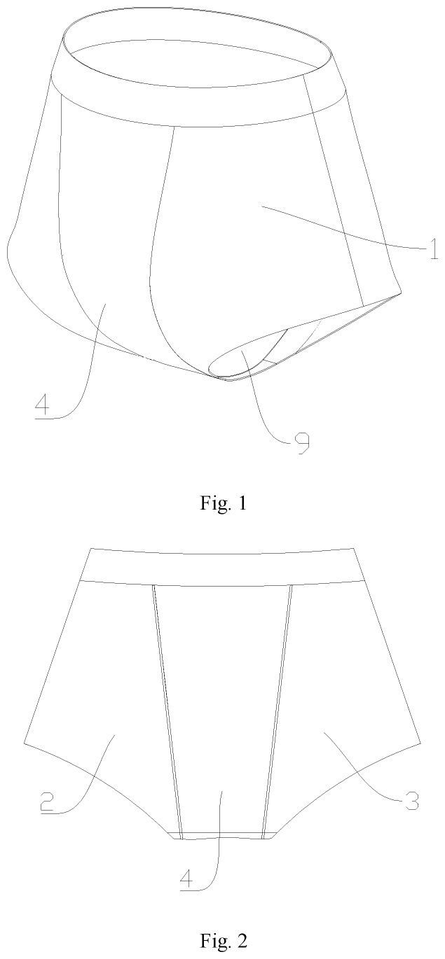

According to an embodiment in the present invention, referring to FIG. 1 and FIG. 2, it provides a sanitary knicker, which includes an underpants body 1 having a first coverage area 2 and a second coverage area 3 at a rear end thereof, the first coverage area 2 is used for fitting a user's left hip, the second coverage area 3 is used for fitting the user's right hip. The sanitary knicker also has a middle crotch cloth 4, a front end of the middle crotch cloth 4 is disposed at a front end of the underpants body 1, a rear end thereof extends toward a rear end of the underpants body 1 to connect with the first coverage area 2 and the second coverage area 3 to wrap a part of the user's left hip, a part of the user's right hip and an area there-between. Due to the rear end of the middle crotch cloth 4 extends toward the rear end of the underpants body 1 to connect with the first coverage area 2 and the second coverage area 3 to wrap a part of the user's left hip, a part of the user's right hip and the area there-between, and the rear end of the middle crotch cloth 4 covers the area between the left hip and the right hip and reaches the waist, resolving the deficiencies that the traditional middle crotch cloth does not extend to the waist, which increases the absorption area of the middle crotch cloth, improves the absorption and leakage prevention effect of the middle crotch cloth so that the user's skin is in a fresh and cool state, which also effectively avoids the middle crotch cloth 4 from being sandwiched between the user's buttocks when the user wears the underpants body 1, the underpants body 1 moves to the middle to shrink with the user's activities. It can also effectively improve the fitting degree between the underpants body 1 and the hip of the user and the area between the hips of the user, thereby improving the comfort of the underwear body 1 when be wore.

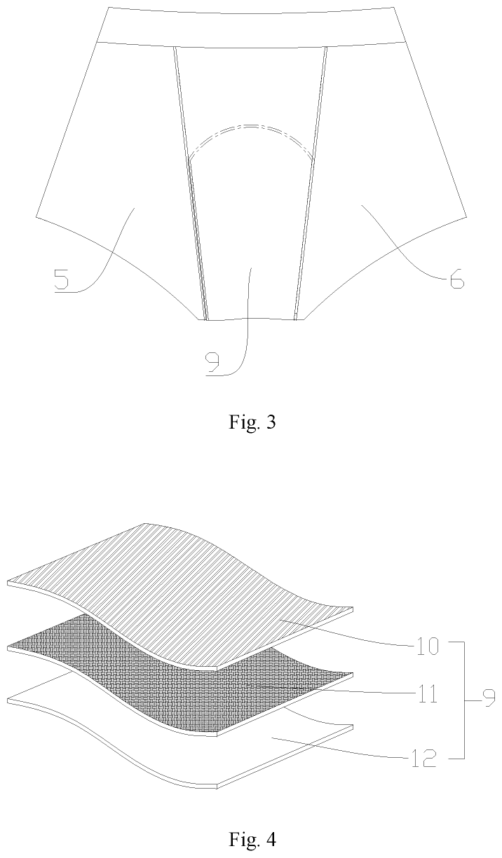

According to an embodiment in the present invention, referring to FIG. 3 and FIG. 4, an inner surface of the middle crotch cloth 4 is provided with an absorption portion 9, which is adhesive to the inner surface of the middle crotch cloth 4, a front of the absorption portion 9 is adhesive to the middle crotch cloth 4 at one half thereof, and a rear end of the absorption portion 9 is adhesive to the waistcloth 13.

Specifically, the absorption portion 9 includes an absorption layer 10, a locking water layer 11 and a leak-proof layer 12, overlapped together. The absorption layer 10 is used to inhale the menstrual blood of the user, the locking water layer 11 is used to adsorb the menstrual blood of the user, and the leak-proof layer 12 is used to isolate the menstrual blood in the locking water layer 11 with other clothing. When the user wears the sanitary knicker, the absorption portion 9 has the function of proofing leakage.

Furthermore, the absorption layer 10, the locking water layer 11 and the leak-proof layer 12 are arranged from a side close to the user's skin to the outside; the absorption layer 10, the locking water layer 11 and the leak-proof layer 12 are overlapped into an integrated structure by an adhesive way so that the absorption layer 10, the locking water layer 11 and the leak-proof layer 12 are not displaced, thereby ensuring the absorption and leakage prevention effect of the absorption portion 9.

According to an embodiment in the present invention, the absorption layer 10 is made from composed of skin-friendly polyester and spandex. The absorption layer 10 is used for inhaling the menstrual blood of the user. The absorption layer 10 is made from 90% polyester and 10% spandex, which has good moisture absorption and can quickly inhale the menstrual blood and ensure the dryness of the middle crotch cloth 4, which also has strong elasticity, is not easy to fold, and will not deform after washing the sanitary knicker for many times.

According to an embodiment in the present invention, the locking water layer 11 is made from skin-friendly polyester, which has good moisture absorption and can better absorb the menstrual blood of the user during the menstrual period. The leak-proof layer 12 is made of a PU membrane, the PU membrane has good waterproof performance, good elasticity and good air permeability, which, used for the leak-proof layer 12, can prevent blood leakage and be breathable, allowing the user's skin in a fresh and cool state, not hot, and the PU membrane has good elasticity, will not be deformed after multiple washing, which can further prolong the service life of the sanitary knicker.

According to an embodiment in the present invention, the absorption layer 10, the locking water layer 11 and the leak-proof layer 12 are adhesive to form the absorption portion 9, the absorption layer 10 can quickly inhale the menstrual blood of the user during the menstrual period, ensuring the fresh and cool state during using the sanitary knicker. The locking water layer 11 is disposed between the absorption layer 10 and the leak-proof layer 12, the locking water layer 11 can further absorb the menstrual blood, and the leak-proof layer 12 can isolate the menstrual blood in the locking water layer 11 so that the menstrual blood is difficult to leak, allowing the user's skin to be in a fresh and cool state. The absorption layer 10 is composed of polyester and spandex, and the locking water layer 11 is made of polyester. The polyester and spandex have good water absorption and facilitate the user to wash the sanitary knicker and realize the reuse of the sanitary knicker. In addition, the absorption portion 9 is easy to clean without worrying about blood residue.

It should be noted that the absorption layer 10, the locking water layer 11 and the leak-proof layer 12 according to the embodiment are not limited to the above material, and the fabric of other materials can be used according to the actual situation, and this is not specifically limited herein.

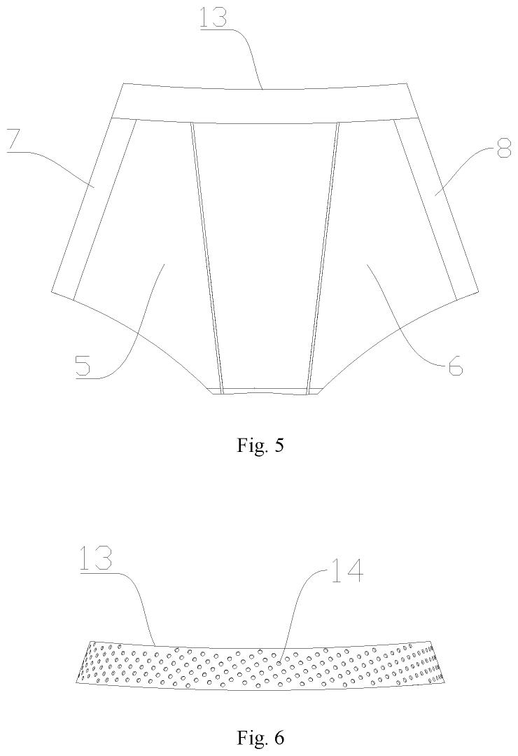

According to an embodiment in the present invention, referring to FIG. 5, the underpants body 1 includes a front-left crotch cloth 5, a front-right crotch cloth 6, a rear-left crotch cloth 7 and a rear-right crotch cloth 8. The front-left crotch cloth 5 is connected with the rear-left crotch cloth 7, and the front-right crotch cloth 6 is connected with the rear-right crotch cloth 8. The front-left crotch cloth 5 and the front-right crotch cloth 6 fit the abdomen of the user, the rear-left crotch cloth 7 is used to fit a part of the left hip of the user, and the rear-right crotch cloth 8 is used to fit a part of the right hip of the user. Both sides of the front end of the middle crotch cloth 4 are respectively connected with the front-left crotch cloth 5 and the front-right crotch cloth 6, and two sides of the rear end of the middle crotch cloth 4 are respectively connected with the rear-left crotch cloth 7 and the rear-right crotch cloth 8 and extend to connect with the waistcloth 13 so that the middle crotch cloth 4 covers the whole back of the underpants body 1, thereby increasing the absorption area of the middle crotch cloth 4, and improving the absorption and leakage prevention effect of the middle crotch cloth 4. The design of the underpants body 1 also resolves the deficiencies that in the rear-left crotch cloth and the rear-right crotch cloth are adhesive in the middle of the traditional underpants body so that the middle crotch cloth can not extend to the back of the traditional underpants body.

According to an embodiment in the present invention, all of the front-left crotch cloth 5, the front-right crotch cloth 6, the rear-right crotch cloth 7, and the rear-right crotch cloth 8 are adhesive to the middle crotch cloth 4 together, forming an integrated structure by the adhesive way, replacing the traditional way of sewing the middle crotch cloth 4 with the front-left crotch cloth 5, the front-right crotch cloth 6, the rear-right crotch cloth 7, the rear-right crotch cloth 8 through needle and thread, what more, there is no suture line between the middle crotch cloth 4 and the front-left crotch cloth 5, the front-right crotch cloth 6, the rear-right crotch cloth 7, the rear-right crotch cloth 8, reducing the friction between the suture line on the user's skin, and making the sanitary knicker more comfortable to wear.

According to an embodiment in the present invention, a left side of the rear-left crotch cloth 7 extends to a side of the front-left crotch cloth 5 and is connected with it, and the right side of the rear-left crotch cloth 7 is connected to the left side of the rear end of the middle crotch cloth 4. The rear-left crotch cloth 7, the middle crotch cloth 4 and the front-left crotch cloth 5 are connected by the adhesive way, therefore there is no suture line between the rear-left crotch cloth 7, the middle crotch cloth 4 and the front-left crotch cloth 5. On one hand, it is beautiful, on the other hand, the surface of the rear-left crotch cloth 7, the middle crotch cloth 4 and the front-left crotch cloth 5 fitting the human body, are flat and flawless, without creating an uncomfortable feeling such as friction or tightening.

According to an embodiment in the present invention, the right side of the rear-right crotch cloth 8 extends to a side of the front-right crotch cloth 6 and connects with it, and the left end of the rear-right crotch cloth 6 is connected to the right side of the rear end of the middle crotch cloth 4. The front-right crotch cloth 6, the middle crotch cloth 4 and the rear-right crotch cloth 8 are connected by the adhesive way, therefore there is no suture line between the front-right crotch cloth 6, the middle crotch cloth 4 and the rear-right crotch cloth 8. On one hand, it is beautiful, on the other hand, the surface of the front-right crotch cloth 6, the middle crotch cloth 4 and the rear-right crotch 8 fitting the human body, are flat and flawless, without creating an uncomfortable feeling such as friction or tightening.

According to an embodiment in the present invention, the rear-left crotch cloth 7 and the rear-right crotch cloth 8 are symmetrically disposed on the underpants body 1. The front-left crotch cloth 5, the front-right crotch cloth 6, the rear-right crotch cloth 7, and the rear-right crotch cloth 8 are all made from composed of nylon and spandex, with the specific proportion being 63% of nylon and 37% of spandex; wherein, the nylon material is smooth, providing a slippery feel to touch, and the nylon material has good abrasion resistance, elasticity resilience, and wrinkle resistance. While the spandex can increase elasticity. A combination of the two is soft, slippery, and has good abrasion resistance, wrinkle resistance, and elasticity. In addition, an adhesive process is applied, so that the menstrual underwear is comfortable and seamless, which can replace most of the sanitary knickers in the market, which are formed by sewing cotton, bamboo fiber, or the like as the main fabric, thereby improving the comfort of the sanitary knicker.

It should be noted that the front-left crotch cloth 5, the front-right crotch cloth 6, the rear-left crotch cloth 7, and the rear-right crotch cloth 8 are not limited to the above material, and the fabric of other materials can be used according to the actual situation, and this is not specifically limited herein.

According to an embodiment in the present invention, referring to FIG. 5-6, the sanitary knicker also includes a waistcloth 13, and the waistcloth 13 is connected to all of the front-left crotch cloth 5, the front-right crotch cloth 6, the rear-left crotch cloth 7, the rear-right crotch cloth 8 and the middle crotch cloth 4 and they are connected by the adhesive way so that they are connected in an integrated structure. It will not leave marks on the user's waist even if it is worn for a long time, which further improves the comfort of the underpants body 1. The waistcloth 13 is provided with plenty of air holes 14, which can increase the air permeability at the waist and avoid the phenomenon of sultry at the waist.

Specifically, the air holes 14 are disposed on the inner of the waistcloth 13 and are rubber strips with holes, and the rubber strips are arranged on the inner side face of the waistcloth 13, the holes on the rubber strip cannot be seen in appearance, and when the waistcloth 13 is stretched, the rubber strip generates a pulling force, thus the holes in the rubber strip can be seen and felt microscopically, and the holes 14 can ventilate the waist during stretching, The phenomenon of sultry at the waist is avoided, thereby improving the comfort level of the waist.

The above mentioned is only a better embodiment of the present invention and is not used to limit the present invention. Any modification, equivalent replacement and improvement made within the spirit and principles of the present invention shall be included in the protection scope of the present invention.

What is claimed is:

1. A sanitary knicker, comprising an underpants body having a first coverage area and a second coverage area at a rear end thereof, said first coverage area is used for fitting a user's left hip, said second coverage area is used for fitting said user's right hip; wherein also comprising a middle crotch cloth, a front end thereof is disposed at a front end of said underpants body, a rear end thereof extends toward said rear end of said underpants body to connect with said first coverage area and said second coverage area, so as to wrap a part of said user's left hip, a part of said user's right hip and an area there-between.

2. The sanitary knicker according to claim 1, wherein an inner surface of said middle crotch cloth has an absorption portion, comprising an absorption layer, a locking water layer and a leak-proof layer, overlapped together; said absorption layer is used to inhale menstrual blood of said user, said locking water layer is used to adsorb said menstrual blood of said user, and said leak-proof layer is used to isolate said menstrual blood in said locking water layer.

3. The sanitary knicker according to claim 2, wherein said absorption layer, said locking water layer and said leak-proof layer are arranged from a side said user's skin to an outside; said absorption layer, said locking water layer and said leak-proof layer are in an integrated structure.

4. The sanitary knicker according to claim 1, wherein said underpants body includes a front-left crotch cloth, a front-right crotch cloth, a rear-left crotch cloth and a rear-right crotch cloth; said front-left crotch cloth is connected with said rear-left crotch cloth, and said front-right crotch cloth is connected with said rear-right crotch cloth, said front-left crotch cloth and said front-right crotch cloth are used to fit said user's abdomen, said rear-left crotch cloth is used to fit a part of said user' left hip, and said rear-right crotch cloth is used to fit a part of said user's right hip.

5. The sanitary knicker according to claim 4, wherein two sides of said front end of said middle crotch cloth are respectively connected with said front-left crotch cloth, said front-right crotch cloth; two sides of said rear end of said middle crotch cloth are respectively connected with said rear-left crotch cloth and said rear-right crotch cloth.

6. The sanitary knicker according to claim 4, wherein said front-left crotch cloth, said front-right crotch cloth, said rear-left crotch cloth and said rear-right crotch cloth are all adhesive to said middle crotch cloth.

7. The sanitary knicker according to claim 4, wherein a left side of said rear-left crotch cloth extends to said front-left crotch cloth and is connected to said front-left crotch cloth, and a right side of said rear-left crotch cloth is connected to a left side of said rear end of said middle crotch cloth.

8. The sanitary knicker according to claim 4, wherein a right side of said rear-right crotch cloth extends to said front-right crotch cloth and is connected to said front-right crotch cloth, and a left side of said rear-right crotch cloth is connected to a right side of said rear end of said middle crotch cloth.

9. The sanitary knicker according to claim 4, wherein said rear-left crotch cloth and said rear-right crotch cloth are symmetrically disposed on said underpants body, said front-left crotch cloth, said front right crotch cloth, said rear-left crotch cloth and said rear-right crotch cloth are all sheet structures.

10. The sanitary knicker according to claim 4, wherein said sanitary knicker also comprising a waistcloth connected to all of said front-left crotch cloth, said front-right crotch cloth, said rear-right crotch cloth, said rear-left crotch cloth and said middle crotch cloth by an adhesive way; said waistcloth is provided with many air holes.
